(19) United States
(12) Patent Application Publication
(10) Pub. No.: US 2022/0406426 A1
(43) Pub. Date: Dec. 22, 2022

(54) CLINICAL DECISION SUPPORT ON CLINICAL ANALYZER

(71) Applicant: Roche Diagnostics Operations, Inc., Indianapolis, IN (US)

(72) Inventors: Florian Schindler, Engelberg (CH); Ian David Parfrement, Unteraegeri (CH); Ulrich Porsch, Weinheim (DE)

(73) Assignee: Roche Diagnostics Operations, Inc., Indianapolis, IN (US)

(21) Appl. No.: 17/893,574

(22) Filed: Aug. 23, 2022

Related U.S. Application Data
(63) Continuation of application No. PCT/EP2021/057165, filed on Mar. 22, 2021.

(30) Foreign Application Priority Data
Mar. 23, 2020 (EP) 20382215.0

(51) Int. Cl. G16H 20/00; G16H 40/63; G16H 80/00; G16H 10/60

(52) U.S. Cl. CPC G16H 20/00; G16H 40/63; G16H 80/00; G16H 10/60

(57) ABSTRACT

A system for providing clinical decision support to a user is presented. The system comprises a Point-of-Care (POC) testing device comprising an integrated clinical decision support module. The POC testing device and the clinical decision support module are connected via a testing module-clinical decision support module interface. The system comprises a testing module between the user and the POC testing device. The testing module requests tests from the user to the POC testing device and sends completed test results from the POC testing device to the user and to the clinical decision support module via the internal interface. The system comprises outside data sources to provide external data via a communication connection to the clinical decision support module of the POC testing device. The clinical decision support module uses the external data and the completed test results to determine and provide clinical decision support information specifically tailored to the user.

### CROSS-REFERENCE TO RELATED APPLICATIONS

This application is a continuation of PCT/EP2021/057165, filed Mar. 22, 2021, which is based on and claims priority to EP 20382215.0, filed Mar. 23, 2020, which are hereby incorporated by reference.

### BACKGROUND

The present disclosure generally relates to clinical decision support modules on a Point-of-Care (POC) device.

A particular type of diagnostic testing is bedside testing or point of care (POC) testing. This type of diagnostic testing is performed mainly by nurses or medical staff primarily trained to operate the instruments available at the site of patient care, such as hospitals, emergency departments, intensive care units, primary care settings, medical centers, patient homes, a physician's office, a pharmacy or a site of an emergency. Major benefits are obtained when the measurement results obtained by a POC testing device(s) are made available immediately/results can be shared instantaneously with all members of the medical team, thereby enhancing communication through decreasing turnaround time.

POC testing devices have become established worldwide and find vital roles in public health. Point-of-Care (POC) testing systems typically have several advantages over large in vitro diagnostic (IVD) systems. POC testing systems can be small, can be easily moved, and can provide fast and accurate results with, in some cases, lifesaving impact. Potential operational benefits of POC testing systems include: faster decision making, reduced operating times, reduced postoperative care time, reduced emergency room time, reduced number of outpatient clinic visits, reduced number of hospital beds required and overall optimal use of professional time.

Generally, the goal of Point of Care systems is to help both healthcare professionals and patients achieve improved clinical and health-economic outcomes, by delivering robust, connected, easy to use point of care solutions outside the central lab, providing immediate results and thus allowing treatment decisions to be made more quickly—inside or outside the hospital. Point of Care testing systems deliver those solutions meeting the clinical need for quick and accurate test results delivered where needed, when needed; on the device, in an electronic healthcare record on a patient/ward monitor, to the clinician on the move and directly to the patient.

Further, POC testing systems have revolutionized patient self-care by allowing a patient (typically, a non-expert user) to perform self-testing and subsequent treatment resulting in large medical and economic advantages.

However, POC testing systems typically have some shortcomings. Namely, POC system testing devices are mostly not capable of providing expert medical advice on the fly and do not fully use all the diagnostic potential of the multiple sources of data provided by each individual patient.

Therefore, there is a need for a clinical decision support system that functions as a module on the POC device itself.

### SUMMARY

According to the present disclosure, a system and method of providing clinical decision support to a user are presented. The method can comprise providing a clinical decision support module in a Point-of-Care (POC) testing device, receiving test results of the user from the POC testing device to the clinical decision support module via an internal interface, receiving external data from a plurality of data sources to the clinical decision support module via a communication connection, determining by the clinical decision support module clinical decision support based on the received test results and external data, and providing the clinical decision support information specifically tailored to the user.

Accordingly, it is a feature of the embodiments of the present disclosure to provide for a clinical decision support system that functions as a module on the POC device itself. Other features of the embodiments of the present disclosure will be apparent in light of the description of the disclosure embodied herein.

### BRIEF DESCRIPTION OF THE SEVERAL VIEWS OF THE DRAWINGS

The following detailed description of specific embodiments of the present disclosure can be best understood when read in conjunction with the following drawings, where like structure is indicated with like reference numerals and in which:

FIG. 1 illustrates schematically a clinical decision support system comprising a POC analyzer with an embedded clinical decision support module according to an embodiment of the present disclosure.

FIG. 2 illustrates schematically a clinical decision support system comprising a POC analyzer with a clinical decision support module and a secondary data feed according to another embodiment of the present disclosure.

### DETAILED DESCRIPTION

In the following detailed description of the embodiments, reference is made to the accompanying drawings that form a part hereof, and in which are shown by way of illustration, and not by way of limitation, specific embodiments in which the disclosure may be practiced. It is to be understood that other embodiments may be utilized and that logical, mechanical and electrical changes may be made without departing from the spirit and scope of the present disclosure.

A system for providing clinical decision support to a user is presented. The system can comprise a Point-of-Care (POC) testing device. The POC testing device can comprise an integrated clinical decision support module that can be embedded in the POC testing device. The POC testing device and the clinical decision support module can be connected via a testing module-clinical decision support module interface. The system can also comprise a test results interface between the user and the POC device. The test results interface can be configured to request tests from the user to the POC device and to send completed test results from the POC device to the user and to the clinical decision support module via the internal interface. The system can also comprise a plurality of diverse data sources configured to provide external data via a communication connection to the clinical decision support module of the POC device. The clinical decision support module can be configured to use the external data and the completed test results to determine and provide clinical decision support information specifically tailored to the user. The clinical decision support information can be displayed on an output display of the POC device.

The term ‘point of care (POC)’ or ‘point of care (POC) environment’ as used herein can be defined to mean a location on or near a site of patient care where medical or medically related services such as medical testing and/or treatment can be provided, including but not limited to hospitals, emergency departments, intensive care units, primary care setting, medical centers, patient homes, a physician's office, a pharmacy or a site of an emergency. The patient care can also be performed in the patient's own residence.

The term ‘communication connection’ as used herein can encompass any type of wireless network, such as a WiFi™, Bluetooth™, GSM™, UMTS or other wireless digital network or a cable based network, such as Ethernet™ or the like. In particular, the communication connection can implement the Internet protocol (IP). For example, the communication connection can comprise a combination of cable-based and wireless networks. The communication connection can also include cloud networking services.

The POC testing device can be an analyzer that can be, for example, handheld or can reside as a benchtop analyzer. Examples of typical POC testing devices can be glucose meters, coagulation meters, blood gas analyzers, imuno-analyzers, and the like.

The term ‘meter’/‘analyzer’/‘analytical instrument’ as used herein can encompass any apparatus or apparatus component configured to obtain a measurement value. An analyzer can be operable to determine via various chemical, biological, physical, optical or other technical procedures a parameter value of the sample or a component thereof. An analyzer may be operable to measure the parameter of the sample or of at least one analyte and return the obtained measurement value. The list of possible analysis results returned by the analyzer can comprise, without limitation, concentrations of the analyte in the sample, a digital (yes or no) result indicating the existence of the analyte in the sample (corresponding to a concentration above the detection level), optical parameters, DNA or RNA sequences, data obtained from mass spectrometry of proteins or metabolites and physical or chemical parameters of various types. An analytical instrument may comprise units assisting with the pipetting, dosing, and mixing of samples and/or reagents. The analyzer may comprise a reagent holding unit for holding reagents to perform the assays. Reagents may be arranged for example in the form of containers or cassettes containing individual reagents or group of reagents, placed in appropriate receptacles or positions within a storage compartment or conveyor. It may comprise a consumable feeding unit.

The analyzer may comprise a process and detection system whose workflow can be optimized for certain types of analysis. Examples of such analyzer are clinical chemistry analyzers, coagulation chemistry analyzers, immunochemistry analyzers, urine analyzers, nucleic acid analyzers, used to detect the result of chemical or biological reactions or to monitor the progress of chemical or biological reactions.

The clinical decision support can comprise providing patient dietary recommendations based on test results of patients' samples, real time medication regime changes such as, for example, insulin, medication adjustments, medication dosing recommendations, treatment recommendations, diagnosis, or combinations thereof.

The terms ‘sample’, ‘patient sample’ and ‘biological sample’ can refer to material(s) that may potentially contain an analyte of interest. The patient sample can be derived from any biological source, such as a physiological fluid, including blood, saliva, ocular lens fluid, cerebrospinal fluid, sweat, urine, stool, semen, milk, ascites fluid, mucous, synovial fluid, peritoneal fluid, amniotic fluid, tissue, cultured cells, or the like. The patient sample can be pretreated prior to use, such as preparing plasma from blood, diluting viscous fluids, lysis or the like. Methods of treatment can involve filtration, distillation, concentration, inactivation of interfering components, and the addition of reagents. A patient sample may be used directly as obtained from the source or used following a pretreatment to modify the character of the sample. In some embodiments, an initially solid or semi-solid biological material can be rendered liquid by dissolving or suspending it with a suitable liquid medium. In some embodiments, the sample can be suspected to contain a certain antigen or nucleic acid.

A further system for providing clinical decision support information to a user is also presented. The system can comprise a Point-of-Care (POC) testing device comprising an integrated clinical decision support module. The POC testing device and the clinical decision support module can be connected via a testing module-clinical decision support module interface. The system can also comprise a testing module between the user and the POC testing device. The testing module can be configured to request tests from the user to the POC testing device and to send completed test results from the POC testing device to the user and to the clinical decision support module via the internal interface. The system can also comprises a plurality of data sources configured to provide external data via a communication connection to the clinical decision support module of the POC testing device. The clinical decision support module can be configured to use the external data and the completed test results to determine and provide clinical decision support information specifically tailored to the user. The system can also comprise a user interface configured to communicate with the user, an internal database integrated within the POC testing device and configured to communicate with the user interface, and a secondary data feed configured to communicate with the user interface and with the communication connection. The secondary data feed can comprise real world user data.

The plurality of diverse outside data sources can comprise data from a variety of different sources such as, for example laboratory information systems (LIS) and/or hospital information systems (HIS) and/or electronic medical records (EMR) data and/or data from the patient himself such as, for example, a continuous glucose monitoring system and/or a device that can monitor vital health signals from the patient such as, for example, heart rate or body temperature.

A ‘data management unit’ or ‘database’ can be a computing unit for storing and managing data. This may involve data relating to biological sample(s) to be processed by the plurality of analyzers in the laboratory or a POC testing device. The data management unit may be connected to an LIS (laboratory information system) and/or an HIS (hospital information system). The data management unit can be a unit within or co-located with a laboratory instrument with the laboratory. It may be part of a control unit. It also may be integrated into the POC testing device. Alternatively, the database may be a unit remotely located. For instance, it may be embodied in a computer connected via a communication connection.

In some instances, the communication connection can be configured to harmonize the external data from the plurality of diverse data sources and to provide a common data protocol to the clinical decision support module in the POC testing device. The external data can be harmonized using any method known by one skilled in the art such as, for example, using data warehouse approaches, ETL (extract, transform, and load) processes, data lakes, data federation, data hubs, and the like.

The system can further comprise a second data feed from the user and an internal database of the POC device. The second data feed from the user can interface with the internal database to acquire historical data. The second data feed can then communicate with the communication connection which, in turn, can provide the second data feed data to the clinical decision support module.

The second data feed can comprise data regarding the quality of a sample used to acquire the test results. For example, the POC device can use a camera to determine if the sample is of adequate quantity and/or quality to be used for testing.

The second data feed can comprise real time user data. The system real time data can comprise dietary data, user health measurements, personal user biomarker data, vital signs of the user such as, for example, pulse rate, twenty-four hour heart rate and the like, user sleep cycles, and combinations thereof.

The system can further comprise an output interface of the POC device. The output interface can be configured to display the clinical decision support information from the clinical decision support module to the user. The clinical decision support information can comprise changes in a testing routine, treatment advice, health prediction information, and combinations thereof. Additionally, the output interface can be configured to display the sample test results to the user.

The system can further comprise sensors incorporated into the POC testing device to capture digital biomarkers from the user for use by the clinical decision support module. The sensors can, for example, include cameras, fingerprint scanners, retinal scanners, breathalyzers, and the like.

A further system for providing clinical decision support information to a user is also presented. The system can comprise a Point-of-Care (POC) testing device comprising an integrated clinical decision support module. The POC testing device and the clinical decision support module can be connected via a testing module-clinical decision support module interface. The system can also comprise a testing module between the user and the POC testing device. The testing module can be configured to request tests from the user to the POC testing device and to send completed test results from the POC testing device to the user and to the clinical decision support module via the internal interface. The system can also comprises a plurality of data sources configured to provide external data via a communication connection to the clinical decision support module of the POC testing device. The clinical decision support module can be configured to use the external data and the completed test results to determine and provide clinical decision support information specifically tailored to the user. The plurality of data sources can comprise laboratory information systems (LIS) and/or hospital information systems (HIS) and/or electronic medical records (EMR) data and/or data from other diagnostic devices. The system can also comprise a user interface configured to communicate with the user, an internal database integrated within the POC testing device and configured to communicate with the user interface, and a secondary data feed configured to communicate with the user interface and with the communication connection. The secondary data feed can comprise real world user data.

A method of providing clinical decision support information to a user is also presented. The method can comprise providing an integrated clinical decision support module in a Point-of-care (POC) testing device, receiving test results of the user from the POC testing device to the clinical decision support module via an internal interface, receiving external data from a plurality of diverse data sources to the clinical decision support module via a communication connection, determining by the clinical decision support module, clinical decision support information based on the received test results and external data, and providing the clinical decision support information specifically tailored to the user.

The method can further comprise displaying the clinical decision support information to the user via an output display of the POC testing device. The output display can also display the test results to the user.

The method can further comprise harmonizing the external data from the plurality of diverse data sources into a common data protocol, and providing the harmonized external data to the clinical decision support module of the POC testing device. The external data can be harmonized using, for example, data warehouse approaches, ETL (extract, transform, and load) processes, data lakes, data federation, data hubs, and the like.

The method can further comprise providing a secondary data feed from the user to the communication connection via the POC testing device. The communication connection can then provide the secondary data to the clinical decision support module. The secondary data feed can provide real time user data as well as historic user data stored in the internal database housed in the POC testing device.

In summary, the present disclosure can directly provide clinical decision support information to the user via an integrated clinical decision support module embedded in a POC testing device; much like a typical app installed on a Smartphone. By having the clinical decision support module embedded in a portable POC testing device, the clinical decision support module can have the capability of using the entirety of the data ecosystem surrounding a patient. In other words, the POC testing device with the embedded clinical decision support module can function as a de facto data hub collecting patient data from multiple interior and exterior sources and using that data as support for the patient's healthcare decisions. The system may also have the capability, by using all the collected data, to recommend changes to the medical testing procedures and/or protocols of the patient.

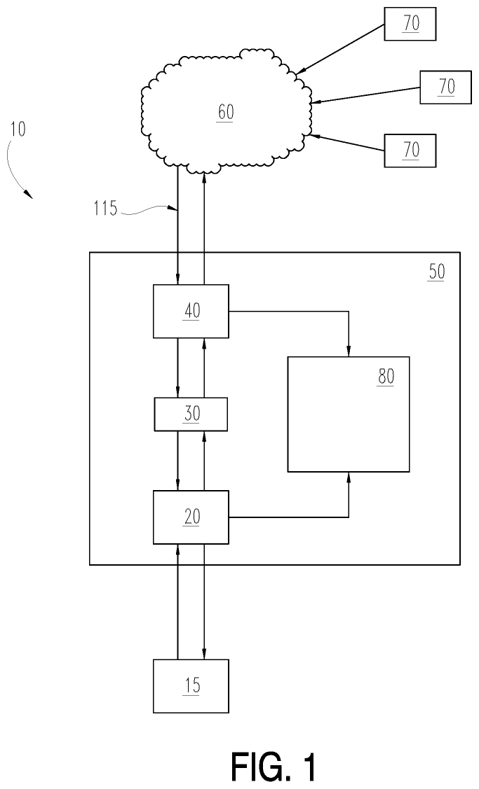

Referring initially to FIG. 1, FIG. 1 schematically illustrates a clinical decision support system 10 comprising a point-of-care (POC) analyzer/testing device 50 with an integrated clinical decision support module 40. The POC testing device 50 can be an analyzer that can be, for example, handheld or can reside as a benchtop analyzer. Examples of typical POC testing devices 50 can be glucose meters, coagulation meters, blood gas meters, imuno-analyzers, and the like.

A user 15 can typically send patient samples to and receive the patient sample test results back from the POC testing device 50 via a testing module 20 embedded in the POC testing device 50. The testing module 20 can be a module housed within the POC testing device 50 that performs the analysis on the patient samples received from the user 15. The user 15 can be any person that has a need to interact with the POC testing device 50. For example, the user 15 can be a medical professional such as, for example, a doctor, nurse, laboratory technician, nurse practitioner, physician's assistant, and the like, that can assist a patient in test taking and/or the user 15 can be a patient himself/herself. The testing module 20 can provide the test results of the patient samples received from the user 15 to the clinical decision support module 40 via a testing module-clinical decision support module 30 as well as provide the test results to the user 15. In one embodiment, the testing module 20 can also interact with an output display 80 of the POC testing device 50 to display the test results to the user 15.

The clinical decision support system 10 can roughly be divided into two main sections. One section can comprise the POC testing device 50 itself along with any embedded modules such as, for example, the testing module 20 and the clinical decision support module 40.

The second section can comprise a communication connection 60 that is communicatively connected to the clinical decision support module 40 via a common data protocol output 115. The communication connection 60 can receive external data that can assist the clinical decision support module 40 in producing the clinical decision support information. The external data can come from a plurality of diverse outside data sources 70 such as, for example, laboratory information systems (LIS) and/or hospital information systems (HIS) and/or electronic medical records (EMR). The plurality of diverse outside data sources 70 can also include clinical systems such as, for example, a continuous glucose monitoring system and other such clinical data sources.

In one embodiment, the communication connection 60 can be capable of harmonizing the external data from the plurality of diverse outside data sources 70 and normalizing the external data from the plurality of diverse outside data sources 70 into a common data protocol output 115 which can then, in turn, be transmitted to the clinical decision support module 40, in order to efficiently support the clinical decision support system 10. The external data can be harmonized using, for example, data warehouse approaches, ETL (extract, transform, and load) processes, data lakes, data federation, data hubs, and the like.

In other words, the clinical decision support system 10 can be thought of as working as a fully functional data hub that can constantly, or at a fixed time, supply the clinical decision support module 40 with real time patient data that can come from a variety of connected devices or from other integrated modules such as, for example, the testing module 20 on the POC testing device 50 or a plurality of diverse outside data sources 70 via the communication connection 60. Examples of such real time patient data can be patient sample test results, dietary patient data, patient health measurements, personal patient biomarker data, vital signs of the patient such as, for example, heart rate, patient sleep cycles, and combinations thereof.

The clinical decision support information formulated by the clinical decision support module 40 can be provided to the user 15 that can be specifically tailored to that particular user 15. In one embodiment, the clinical decision support information can be displayed to the user 15 on the output display 80. The clinical decision support information can comprise, for example, providing patient dietary recommendations based on test results of patients' samples, real time medication regime changes such as, for example, insulin, adjustments, medication dosing recommendations, treatment recommendations, diagnosis, health prediction information or combinations thereof.

Further, all of the patient data that is supplied to the clinical decision support system 10 through the various means can also be used to improve the clinical decision support for other patients using other POC testing devices in the clinical decision support system 10 if all of the patient data is properly anonymized. In other words, the anonymized patient data from one POC testing device can be one of the plurality of diverse outside data sources transmitted to another POC testing device.

In one embodiment, the clinical decision support system 10 may advise the user 15 that a patient may need change his/her testing routine based on advice by a clinical advisor, i.e., real time data supply. In this embodiment, the clinical decision support information provided to the user 15 may be used to alter the testing routine of a patient. In another embodiment, the clinical decision support information provided to the user 15 can be patient treatment advice or may be even health prediction information.

In one embodiment, one of the plurality of diverse outside data sources 70 can be a glucose monitoring system. In this embodiment, the clinical decision support information can be, for example, insulin-dosing recommendations. This clinical decision support can be quite useful in clinical glucose management as well as for patient self-care. For example, if the patient uses an insulin pump as part of his/her glucose monitoring system, the clinical decision support information can be used to help control the insulin pump on the fly. In addition, the clinical decision support system can also use the clinical data supplied by the patient and the other diverse outside data sources to provide additional advice that is not necessarily related to the patient's medical self-treatment such as, for example, recommendations for different dietary options such as, for example, low carbohydrate and/or high protein and/or plant-based and/or gluten-free diets.

In another embodiment, the clinical decision support system 10 could be part of a coagulation-testing regime. In this case, several outside factors may influence the coagulation status of the patient. For example, the clinical decision support system 10 can use not only previous coagulation patient data from an internal database 90 or from other external data sources to assist with developing the clinical decision support information but the clinical decision support system 10 can also access other sources of data such as, for example, the patient's diet log, if such a log is kept by the patient, as well as the patient's medication log. By combining previous coagulation patient data with diet and medication logs, the clinical decision support information for a coagulation-testing regime can provide tailored advice directly to that particular patient.

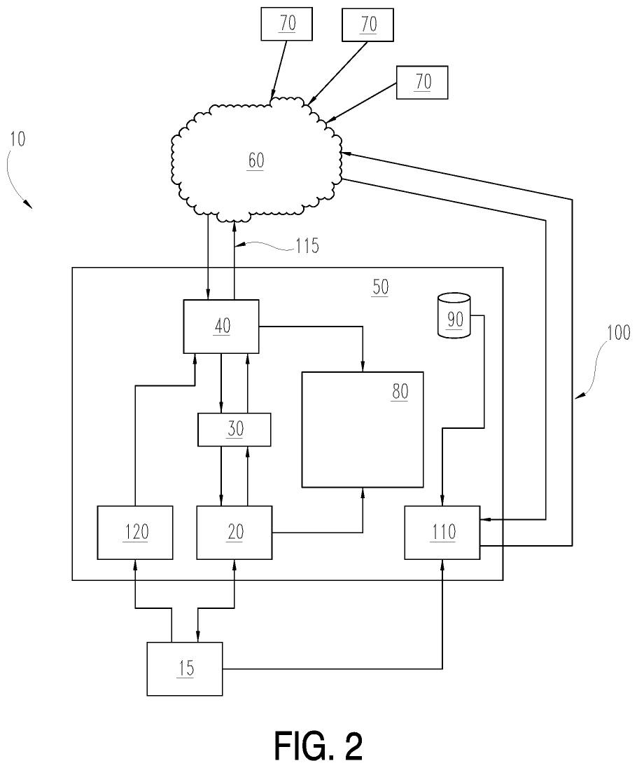

In another embodiment, illustrated schematically in FIG. 2, a clinical decision support system 10 comprising a POC analyzer/testing device 50 with an integrated clinical decision support module 40 and a secondary data feed 100. This embodiment is similar to the embodiment depicted in FIG. 1 and uses the same reference numbers for the same features.

However, the embodiment depicted in FIG. 2 comprises a secondary data feed 100 and an internal database 90 integrated into the POC testing device 50. The secondary data feed 100 can communicate with a user interface 110 of the POC testing device 50. The user interface 110 can receive real time data from the user 15 as well as historical data from the internal database 90. The user interface 110 can then communicate with the communication connection 60 via the secondary data feed 100. The secondary data feed 100 can be thought of an additional external data source. The communication connection 60 can then provide this secondary data feed 100 information to the clinical decision support module 40 to assist in providing the clinical decision support information to the user 15.

In one embodiment, the secondary data feed 100 can comprise patient real world data and can come directly from the user/patient 15. The patient real world data can comprises, for example, dietary data, health measurements, and even biomarker data from the patient personal medical history.

In another embodiment, the patient biomarker data can be acquired via sensors 120 incorporated in the POC testing device 50. The sensors 120 of the POC testing device 50 can comprise cameras, retinal scanners, fingerprint scanner, breathalyzers, just for example. Other sensors 120 can also be envisioned and used. This patient real world data can then be provided via the secondary data feed 100 and can be used by the clinical decision support module 40 to provide clinical decision support specifically tailored to the user 15.

In another embodiment, the secondary data feed 100 can comprise data regarding the quality and/or quantity of a sample used to acquire the test results. For example, this quality/quantity data can be acquired by a camera integrated into the POC testing device 50 in manner similar to acquiring biomarker data.

Further disclosed and proposed is a computer program including computer-executable instructions for performing the method according to the present disclosure in one or more of the embodiments enclosed herein when the program can be executed on a computer or computer network. Specifically, the computer program may be stored on a computer-readable data carrier. Thus, specifically, one, more than one or even all of method steps as disclosed herein may be performed by using a computer or a computer network, preferably by using a computer program.

Further disclosed and proposed is a computer program product having program code, in order to perform the method according to the present disclosure in one or more of the embodiments enclosed herein when the program is executed on a computer or computer network. Specifically, the program code may be stored on a computer-readable data carrier.

Further disclosed and proposed is a data carrier having a data structure stored thereon, which, after loading into a computer or computer network, such as into a working memory or main memory of the computer or computer network, may execute the method according to one or more of the embodiments disclosed herein.

Further disclosed and proposed is a computer program product with program code stored on a machine-readable carrier, in order to perform the method according to one or more of the embodiments disclosed herein, when the program is executed on a computer or computer network. As used herein, a computer program product refers to the program as a tradable product. The product may generally exist in an arbitrary format, such as in a paper format, or on a computer-readable data carrier. Specifically, the computer program product may be distributed over a data network.

Further disclosed and proposed is a modulated data signal, which contains instructions readable by a computer system or computer network, for performing the method according to one or more of the embodiments disclosed herein.

Referring to the computer-implemented aspects of the present disclosure, one or more of the method steps or even all of the method steps of the method according to one or more of the embodiments disclosed herein may be performed by using a computer or computer network. Thus, generally, any of the method steps including provision and/or manipulation of data may be performed by using a computer or computer network. Generally, these method steps may include any of the method steps, typically except for method steps requiring manual work, such as providing the samples and/or certain aspects of performing measurements.

Further disclosed and proposed is a computer or computer network comprising at least one processor, wherein the processor is adapted to perform the method according to one of the embodiments described in this description.

Further disclosed and proposed is a computer loadable data structure that is adapted to perform the method according to one of the embodiments described in this description while the data structure is being executed on a computer.

Further disclosed and proposed is a storage medium, wherein a data structure is stored on the storage medium and wherein the data structure is adapted to perform the method according to one of the embodiments described in this description after having been loaded into a main and/or working storage of a computer or of a computer network.

It is noted that terms like “preferably,” “commonly,” and “typically” are not utilized herein to limit the scope of the claimed embodiments or to imply that certain features are critical, essential, or even important to the structure or function of the claimed embodiments. Rather, these terms are merely intended to highlight alternative or additional features that may or may not be utilized in a particular embodiment of the present disclosure.

Having described the present disclosure in detail and by reference to specific embodiments thereof, it will be apparent that modifications and variations are possible without departing from the scope of the disclosure defined in the appended claims. More specifically, although some aspects of the present disclosure are identified herein as preferred or particularly advantageous, it is contemplated that the present disclosure is not necessarily limited to these preferred aspects of the disclosure.

We claim:

1. A system for providing clinical decision support information to a user, the system comprising:
a Point-of-Care (POC) testing device comprising an integrated clinical decision support module, wherein the POC testing device and the clinical decision support module are connected via a testing module-clinical decision support module interface;
a testing module between the user and the POC testing device, wherein the testing module is configured to request tests from the user to the POC testing device and to send completed test results from the POC testing device to the user and to the clinical decision support module via the internal interface;
a plurality of data sources configured to provide external data via a communication connection to the clinical decision support module of the POC testing device, wherein the clinical decision support module is configured to use the external data and the completed test results to determine and provide clinical decision support information specifically tailored to the user;
a user interface configured to communicate with the user;
an internal database integrated within the POC testing device and configured to communicate with the user interface; and
a secondary data feed configured to communicate with the user interface and with the communication connection, wherein the secondary data feed comprises real world user data.

2. The system according to claim 1, wherein the POC testing device is one of: a glucose meter, a coagulation meter, a blood gas analyzer, or an immune-analyzer.

3. The system according to claim 1, wherein the clinical decision support information comprises patient dietary recommendations based on test results, real time medication adjustments, medication dosing recommendations, treatment recommendations, diagnosis, or combinations thereof.

4. The system according to claim 1, wherein the plurality of data sources comprises laboratory information systems (LIS) and/or hospital information systems (HIS) and/or electronic medical records (EMR) data and/or data from other diagnostic devices.

5. The system according to claim 1, wherein the communication connection is configured to harmonize the external data from the plurality of data sources and to provide a common data protocol output to the clinical decision support module .

6. The system according to claim 1, wherein the secondary data feed comprises data regarding the quality and/or quantity of a sample used to acquire the test results of the patient.

7. The system according to claim 1, wherein the real world data comprises patient dietary data, patient health measurements, personal patient biomarker data, vital signs of the patient, patient sleep cycles, and combinations thereof.

8. The system according to claim 1, further comprising,
an output display integrated into the POC testing device, wherein the output display is configured to display the clinical decision support information from the clinical decision support module and is configured to display test results to the user.

9. The system according to claim 10, wherein the clinical decision support information comprises changes in a testing routine, treatment advice, health prediction information, and combinations thereof.

10. The system according to claim 1, further comprising,
sensors incorporated into the POC testing device to capture digital biomarkers from the user for use by the clinical decision support module.

11. A method of providing clinical decision support to a user, the method comprising:
providing a clinical decision support module in a Point-of-Care (POC) testing device;
receiving test results of the user from the POC testing device to the clinical decision support module via an internal interface;
receiving external data from a plurality of data sources to the clinical decision support module via a communication connection;
determining by the clinical decision support module clinical decision support based on the received test results and external data; and
providing the clinical decision support information specifically tailored to the user.

12. The method according to claims 13, furthering comprising,
harmonizing the external data from the plurality of data sources into a common data protocol output; and
providing the harmonized external data to the clinical decision support module.

13. The method according to claim 13, further comprising,
providing a secondary data feed from the user to the communication connection, wherein the secondary data feed provides real world user data.
